(19) United States
(12) Patent Application Publication
(10) Pub. No.: US 2012/0225420 A1
(43) Pub. Date: Sep. 6, 2012

(54) BIO VAPOR STIMULATION SYSTEM

(76) Inventors: Tudor D. WILLIAMS, South Pasadena, CA (US); Evan Williams, Palos Verdes, CA (US)

(21) Appl. No.: 13/108,609

(22) Filed: May 16, 2011

Related U.S. Application Data
(60) Provisional application No. 61/345,012, filed on May 14, 2010.

(51) Int. Cl. C12Q 3/00

(52) U.S. Cl. 435/3

(57) ABSTRACT

The organic material in a landfill's incoming solid waste is converted into a useful biogas (methane). A bio vapor stimulation system comprises components for mixing bacteria and nutrients and for growing bacteria, as well as process sensors, and a delivery system to provide an appropriate balance of: (1) anaerobic bacteria, (2) nutrients, and (3) humidification. Measurements of landfill site conditions, landfill gas, landfill gas condensate for temperature, pH, alkalinity, COD/BOD and gas composition, oxidation-reduction potential (ORP), volatile acid concentration, and/or other parameters are used as process control inputs. Based upon these measured indicators of the health and status of the anaerobic bacteria community within the site, various process changes may be administered such as bacteria composition, additives, nutrient composition and quantity, and temperature and ph of the liquid being fed into a carrier gas for site humidification, thereby facilitating a more efficient conversion of organic waste into biogas.

### CROSS REFERENCE TO RELATED APPLICATIONS

This application claims benefit of and priority from U.S. Provisional Application US 61/345,012 filed on 14 May 2010.

### BACKGROUND

The moisture content within a landfill's incoming solid waste is usually sufficient to provide total conversion of the organic material into methane. However, the moisture is not distributed evenly and under certain solid waste practices, it is extracted without replacement, thus leaving the waste in place unable to be biologically converted into methane until a much later date when maintenance funds run out and the solid waste is introduced to rain water via the process of site cover erosion and water intrusion.

The anaerobic conversion of waste into methane requires a number of bacteria families and a number of process steps to break down the solid waste into components and gases which can be converted by methanagenes into methane. The better the environment in terms of bacteria families, nutrients, and micro nutrients, temperature, alkalinity/pH, and humidity, the more efficient the conversion of the organic waste in terms of the amount of waste converted and the rate of waste conversion.

FIG. 1 provides an illustration of the pathways through which organic material in a landfill can be converted into methane. Within the anaerobic food chain, complex organic compounds are degraded by different groups of bacteria through a variety of anaerobic or fermentation biochemical reactions. These reactions result in the production of soluble and simplistic organic compounds. As one group of bacteria provides soluble compounds, they are quickly degraded as substrate by another group of bacteria. For methane production, the compounds must be degraded to simplistic organic and inorganic compounds that can be used as substrate by methane-forming bacteria. These compounds include the organics formate, methanol, methylamine, and acetate and the inorganics hydrogen and carbon dioxide.

The anaerobic food chain consists of several groups of facultative anaerobes and anaerobes that degrade and transform complex organic compounds into simplistic organic compounds. The final organic compound produced in the anaerobic food is methane. This compound is the most reduced form of carbon.

Methane is produced by methane-forming bacteria from organic compounds such as acetate (equation 1) or from the combination of the inorganics carbon dioxide (as bicarbonate (HCO₃) or carbonate (CO₃ with hydrogen (H₂ (equations 2 and 3).

CH₃ COOH→CH₄+CO₂ (1)

4H₂+HCO₃+H⁺→CH₄+3 H₂O (2)

4H₂+CO₃+2H⁺→CH₄+3H₂O (3)

These organisms consume hydrogen with carbon dioxide to produce methane. There are three principle groups of methane-forming bacteria. These groups are (1) the hydrogenstrophic methanagenes, (2) the acetotrophic methanogens, and (3) the methylotrophic methanogens. The term “tropic” refers to the substrate used by the bacteria. Table 1 illustrates some substrates used by methane-forming bacteria. Table 2 illustrates various species of methane-forming bacteria and their substrates. Within the anaerobic food chain, there are syntrophic relationships between bacteria. In these relationships, at least two different bacteria are involved and the activity of one organism is dependent on the activity of another organism. An example of a syntrophic relationship in the anaerobic food chain is the association between hydrogen-producing bacteria and hydrogen-consuming bacteria. In this association, hydrogen-producing bacteria degrade organic compounds to more simplistic compounds and hydrogen (equation 4).

Glucose+4H₂O→2 acetate+2HCO3+2H⁺+4H₂ (4)

Bacteria degrade substrates through the use of enzymes. Enzymes are proteinaceous molecules that catalyze biochemical reactions. Two types of enzymes are involved in substrate degradation: endoenzymes and exoenzymes (FIG. 2). Endoenzymes are produced in the cell and degrade soluble substrate within the cell. Exoenzymes also are produced in the cell but are released through the “slime’ coating the cell to the insoluble substrate attached to the slime. Once in contact with the substrate the exoenzyme solubilize particulate and colloidal substrates. Once solubilized, these substrates enter the cell and are degraded by endoenzymes.

TABLE 1
Substrates Used by Methane-Forming Bacteria

| Substrate | Chemical Formula |
|---|---|
| Acetate | CH₃ COOH |
| Carbon dioxide | CO₂ |
| Carbon Monoxide | CO |
| Formate | HCOOH |
| Hydrogen | H₂ |
| Methanol | CH₃OH |
| Methylamine | CH₃NH₂ |

TABLE 2
Species of methane-Forming Bacteria and Their Substrate

| Species | Substrate |
|---|---|
| Methanobacterium formiclum | Carbon dioxide, formate, hydrogen |
| Methanobacterium | Hydrogen, CO₂, CO |
| thermoantotrophicum | |
| Methanococcus frisius | Hydrogen, methanol, methyglamine |
| Methanococcus mazel | Acetate, methanol, methyglamine |
| Methanosarcina bakeril | Acetate, CO₂, H₂, methanol, |
| | methylamine |

### DISCLOSURE OF INVENTION

A bio vapor stimulation system preferably comprises components for mixing bacteria and nutrients and for growing bacteria, as well as process sensors, and a novel delivery system to provide an appropriate balance of: (1) anaerobic bacteria, (2) nutrients, and (3) humidification in a carrier gas for deposition through condensation, into selected volumes of a landfill (or other coal or biomass resources), as determined by various sensors. Measurements of the landfill site conditions, the landfill gas, the landfill gas condensate for temperature, pH, alkalinity, COD/BOD, gas composition; oxidation-reduction potential (ORP), volatile acid concentration, and/or other parameters may be used as process control inputs. Based upon these measured indicators of the health and status of the anaerobic bacteria community within the site, various process changes may be administered such as bacteria composition, additives, nutrient composition and quantity, temperature and ph of delivered liquid feed into carrier gas for the site humidification, to establish the restoration of an efficient biologic environment for the anaerobic conversion of organic waste or other carbonaceous materials.

Such a bio vapor stimulation of a landfill or other waste provides: (1) The stabilization (reduction of organic waste) of the landfill via the organic conversion of waste in a controlled manner during the economic life of the landfill site (or other waste formation), (2) the development of methane gas from the waste, for a variety of energy and other beneficial uses, (3) the sequestration of carbon via biologic conversion to methane, (4) an increase in air space on the landfill for the inclusion of more waste, thus reducing the total landfill footprint for solid waste storage, and (5) utilization of specific natural bacteria to improve the gas quality via reducing hydrogen sulfide production or increasing the conversion of more challenging wastes, such as lignin, etc.

Bio vapor stimulation provides an improved climate for the anaerobic conversion of the solid waste stream with a solution of temperature controlled nutrient and bacteria enriched water supplemented with micro-nutrients and alkalinity control to consume and convert both the enriched water vapor into methane in combination with the balanced bacteria biologic consumption of the solid waste into methane gas. In that fashion, the vapor addition will equate to bacteria growth and solid waste reduction through a family of biologic steps leading to methane production, without the formation of a hydraulic column.

### DRAWINGS

FIG. 1 illustrates the pathways through which organic material can be converted into methane.

FIG. 2 depicts digestion characteristics of two types of enzymes involved in substrate degradation.

FIG. 3 shows digestion characteristics of two types of methane-forming bacteria.

FIG. 4 depicts an exemplary bacteria/bio-nutrient measurement, mixing heating, filtering, and pumping equipment.

FIG. 5 depicts an exemplary carrier gas and bacteria/bio-nutrient mixing and injection system, comprising front view 5A, side view 5B, and detailed partial views 5C, 5D, 5E.

FIG. 6 is a temperature-enthalpy T-H chart.

FIG. 7 shows reduction in gas temperature as a function of water temperature prior to mixing.

FIG. 8 illustrates temperature profiles of two landfills.

FIGS. 9 & 10 illustrate exemplary condensation percentages from two primary carrier gases

FIGS. 11 & 12 show initial carrier gas temperatures

FIG. 13 shows an exemplary injector/well pattern and control system

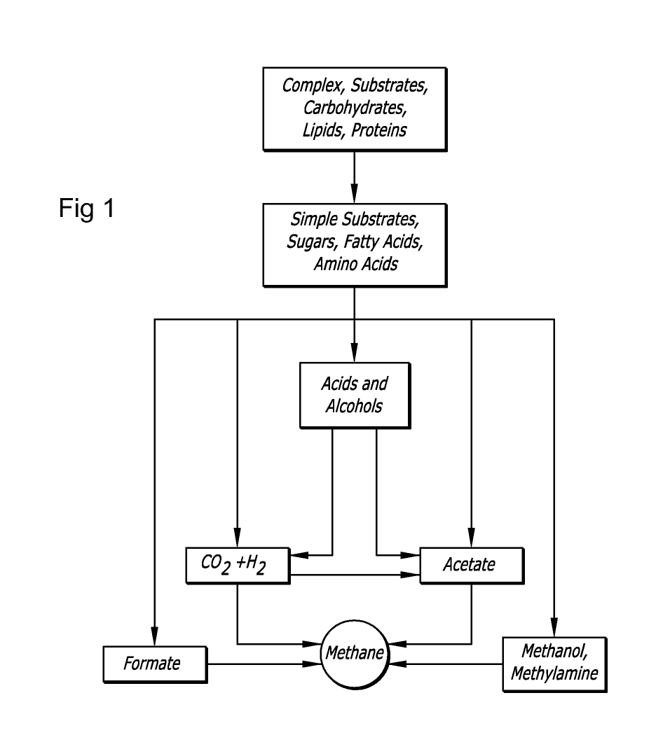

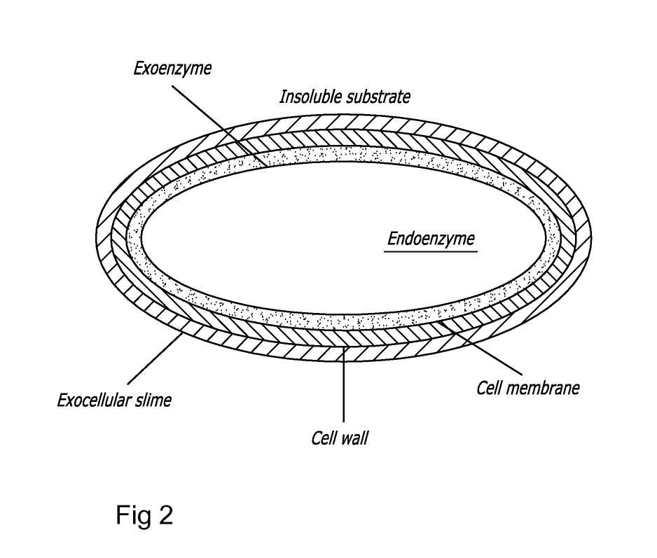

### DISCLOSURE OF PREFERRED EMBODIMENT(S)

The desired degradation of organic compounds by hydrogen-producing bacteria occurs only if the partial pressure of hydrogen can be kept low, <10⁻⁴ atmosphere.

No single bacterium produces all the exoenzymes that are needed to degrade the large variety of particulate and colloidal substrates that are found in solid waste and other biomasses. Each exoenzyme, as well as each endoenzyme, degrades only a specific substrate or group of substrates. Therefore, a large and diverse community of bacteria is needed to ensure that the proper type of exoenzymes and endoenzymes are available for degradation of the substrate present. Table 3 illustrates the enzyme required for certain substrates and Table 4 illustrates how enzymes are utilized in the major pathway for methane production from solids.

Methane gas production by anaerobic bacteria is facilitated by coenzymes. Coenzymes are metal-laden organic acids that are incorporated into enzymes and allow the enzymes to work more efficiently. The coenzymes are components of energy-producing electron transfer systems that obtain energy for the bacterial cell and remove electrons from degraded substrates. Coenzymes are used to reduce carbon dioxide (CO₂) to methane. The nickel-containing coenzymes are important hydrogen carriers in methane-forming bacteria.

There are two type of enzymes that are used to degrade substrates. Exoenzymes are produced in the cell and released through the cell membrane and cell wall to hydrolyze insoluble substrates that have been adsorbed by the exocellular slime coating the cell. Soluble wastes enter the bacteria cell and are degraded by endoenzyme. To perform at their optimal conversion, these bacteria need to have a balanced nutrient and micro nutrient supply.

TABLE 3
Exoenzymes and Substrates

| Substrate to be | Exoenzyme | | | |
|---|---|---|---|---|
| degraded | Needed | Example | Bacteria | Product |
| Polysaccharides | Saccharolytic | Cellulose | Cellulomonas | Simple |
| | | | | Sugar |
| Proteins | Proteclytic | Protease | Bacillus | Amino Acids |
| Lipids | Lipolytic | Lipase | Mycobacterium | Fatty Acids |

TABLE 4
Enzymes Used in the Three Stages of Anaerobic Digestion of Solids

| Stage | Activity | Enzyme Used |
|---|---|---|
| First | Hydrolysis: solubilization of particulate | Exoenzymes |
| | and colloidal waste | |
| Second | Acid-forming: conversion of soluble | Endoenzymes |
| | organic acids and alcohols to | |
| | acetate, carbon dioxide, and | |
| | hydrogen | |
| Third | Methanogenesis: production of | Endoenzymes |
| | methane and carbon dioxide | |

To perform close to their optimal conversion, these bacteria need to have a balanced nutrient and micro nutrient supply.

Anaerobic Bacteria Nutrients and Micro Nutrients

The chemical composition of bacteria cells is shown in Table 5. To keep the cell structure healthy, the two macronutrients of most importance are nitrogen and phosphorus. These nutrients, like all nutrients, are available to bacteria only in soluble form. These forms are ammonical-nitrogen (CH₄⁺—N) and orthophosphate-phosphorus (HPO⁺₄—P). Some methane bacteria can obtain nitrogen from other sources. If it is determined based upon chemical oxygen demand (COD) measurement on the biogas condensate or leachate that nitrogen is needed, then nitrogen can be supplied by the addition of either ammonium chloride, aqueous ammonia, or urea. If phosphorous additions are required, phosphate salts, and phosphoric acid may be used.

TABLE 5
Elementary Composition of Bacterial Cells (Dry Weight)

| Element | Approximate Percent Composition |
|---|---|
| Carbon | 50 |
| Oxygen | 20 |
| Nitrogen | 12 |
| Hydrogen | 8 |
| Phosphorus | 2 |
| Sulfur | 1 |
| Potassium | 1 |
| Others | 6 |

Because methane-forming bacteria possess several unique enzyme systems, they have micronutrient requirements that are different from those of other bacteria. They need several micronutrients, especially cobalt, iron, nickel, and sulfide, as well as trace components of selenium and tungsten. Yeast extract can be used to supply these micronutrients and the amino acids cysterine and methronime can be used to provide sulfide which is the source of sulfur, for methane-forming bacteria.

The temperature at which bacteria operates is a significant factor in the rate at which carbonaceous material is transformed into methane. A increase in temperature results in more enzymatic activity or reactions for the anaerobic bacteria food chain. Various methane bacteria also become dominate at certain temperature ranges, therefore, an understanding and measurement of this process variable is an important aspect of the control of the bio-methane conversion process.

Anaerobic Bacteria and Temperature

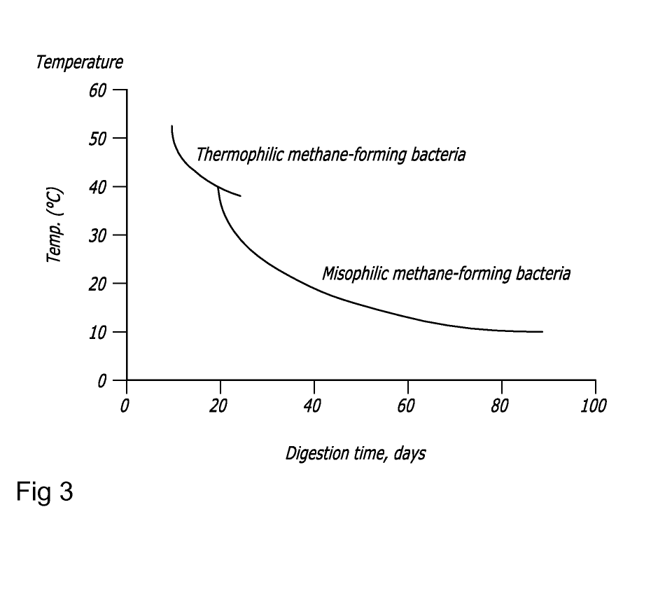

Methane-forming bacteria can function over a wide temperature range, however, most methane-forming bacteria are active in two temperature ranges. These ranges are the mesophilic range from 30° C. to 35° C. (86° F. to 95° F.) and the thermophilic range from 50° C. to 60 C (122° F. to 140° F.). Methane production can occur over a wide temperature range with digestion efficiency improving with higher temperature predominant methane-forming bacteria as shown in FIG. 3. Table 6 illustrates methane-forming bacteria families that are predominant as a function of the substrate and local temperature. Therefore, the biomass temperature is a good indication of what methane-forming bacteria are operating and the potential efficiency of biomass conversion to methane, as well as the type of methane bacterial families that should be introduced into a certain temperature zone within the biomass.

TABLE 6
Temperature Range for Methane Production for
Municipal Anaerobic Digesters

| Bacteria Group | Temperature Range ° C. |
|---|---|
| Psychophiles | 5-15 |
| Mesophiles | 30-35 |
| Thermophiles | 50-60 |
| Hyperthermophiles | >65 |

Anaerobic Bacteria and PH/Alkalinity Levels

Acceptable enzymatic activity of acid-forming bacteria occurs above pH 5.0, but acceptable enzymatic activity of methane-forming bacteria does not occur below pH 6.2. Most anaerobic bacteria, including methane-forming bacteria, perform well within a pH range of 6.8 to 7.2. Table 7 illustrates the optimum pH range for growth of some methane-forming bacteria.

Methane production occurs over a relatively large range of temperature values. Due to increased enzyme reactions, the higher the temperature, the faster the waste is consumed and methane produced.

Sufficient alkalinity is essential for process pH control. Alkalinity serves as a buffer that prevents rapid changes in pH. The digestion process of anaerobic bacteria is enhanced by high alkalinity concentration because methane-forming bacteria require bicarbonate alkalinity. Chemicals that release bicarbonate-alkalinity directly are preferred. Table 8 presents chemicals commonly used for alkalinity addition. Although pH of the bio-mass is more easily and quickly determined than the alkalinity of the bio-mass, the pH is only an indication of what has already happened in the anaerobic digestion process, whereas changes in alkalinity indicate what is happening in the anaerobic digestion process within the biomass.

TABLE 7
Optimum Growth pH of Some Methane-Forming Bacteria

| Genus | pH |
|---|---|
| Methanosphaera | 6.6 |
| Methanoherms | 6.5 |
| Methanogenium | 7.0 |
| Methanolacinia | 6.8-7.2 |
| Methanomicrobuim | 6.1-6.9 |
| Methanospirillum | 7.0-7.5 |
| Methanohaloblium | 6.5-7.5 |
| Methanolobus | 6.5-6.8 |
| Methanthrix | 7.1-7.6 |

TABLE 8
Chemicals Commonly Used for Alkalinity Addition

| Chemical | Formula | Buffering === |
|---|---|---|
| Sodium bicarbonate | NaHCO₃ | NA⁺ |
| Potassium tricarbonate | KHCO₃ | K⁺ |
| Sodium carbonate (soda | Na₂ CO₃ | Na⁺ |
| ash) | | |
| Potassium carbonate | K₂ CO₃ | K⁺ |
| Calcium carbonate | Ca CO₃ | CA³⁺ |
| Anhydrous ammonia | NH₃ | NH⁺ |
| Sodium Nitrate | Na NO₃ | Na⁺ |

Bacteria for Special Functions of Biomass Conversion

Certain families of bacteria can be used to perform specific functions within the solid waste or biomass. Some of the substrate produced in the anaerobic food chain are organic and some are inorganic. Bacteria that respire by using organic substrates are organotrophs and bacteria that respire using inorganic substrates are chemolithotrophs. Several important groups of chemolithotrophs that can perform beneficial functions in the process of forming methane are shown in Table 9. These bacteria groups include ammonium oxidizers, hydrogen bacteria, iron bacteria, nitrite oxidizers, and sulfur bacteria. There are even bacteria which with an electric current, can directly convert CO₂ to methane and some which can break down lignin. Some of these bacteria families can be used to reduce odor (H₂S), and improve total bio-mass conversion and methane production.

TABLE 9
Special Function Chemolithotrophs

| Group | Substrate | Product |
|---|---|---|
| Ammoniium oxidizers | NH₄ | NO₂ |
| Hydrogen bacteria | H₂ | H⁺ |
| Iron bacteria | FE²⁺ | FE³⁺ |
| Nitrate oxidizers | NO₂ | NO₃ |
| Sulfur bacteria | H2S | S⁺ |
| | S | SO_{2/3} |
| | SO_{2/3} | SO_{2/4} |

Bio Vapor Stimulation Technology Components

The bio vapor stimulation process comprises at least some, and preferably all, of the following steps:
Ingredient selection of bacteria, nutrients, etc.;
Temperature adjustment (heat addition) and ingredient addition (bacteria families, nutrients, micro nutrients, alkalinity, etc.) to aqueous mixing chamber;
Temperature adjustment and growth of bacteria families in holding tank.
Filtration of bacteria growth chamber effluent;
Transport of bacteria and nutrient from holding tank to injector via transfer pump;
Carrier gas acquisition and pressurization;
Carrier gas heat addition;
Injection and mixing of carrier gas and bio-nutrient mixture;
Monitor landfill site for temperature and moisture;
Test extraction well condensate in injector influenced area for COD/BOD/pH, and other biologic parameters;
Adjust process ingredients and process variables, if required.

Each of these process steps has a variety of implementation options as a function of the biomass area being treated and existing infrastructure at the landfill, biomass, or coal seam site.

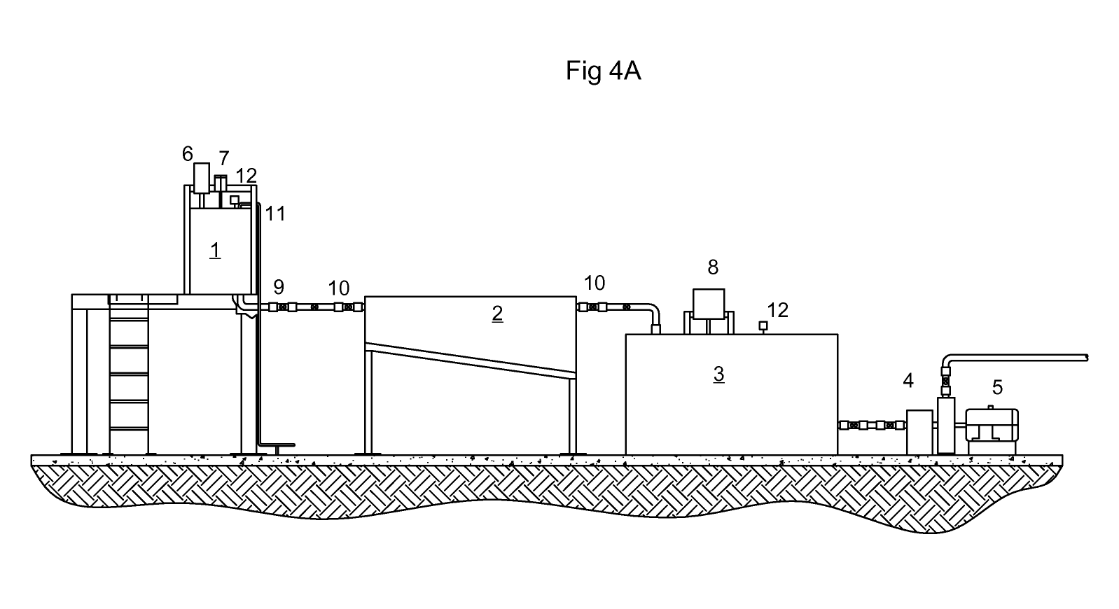

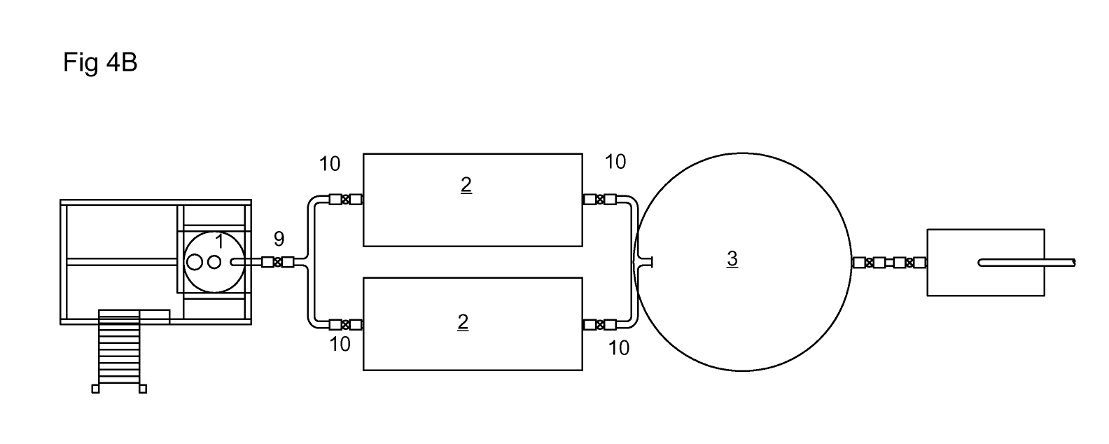

FIG. 4 depicts exemplary bacteria/bio-nutrient measurement, mixing heating, filtering, and pumping equipment, including bacteria blending tank 1, gravity separator 2, bacteria growth tank 3, pump suction filter 4, centrifugal pump 5, bacteria dispenser 6, auger 7, nutrient dispenser 8, flow control valve 9, switching valves 10, water supply valve 11, and level sensor 12.

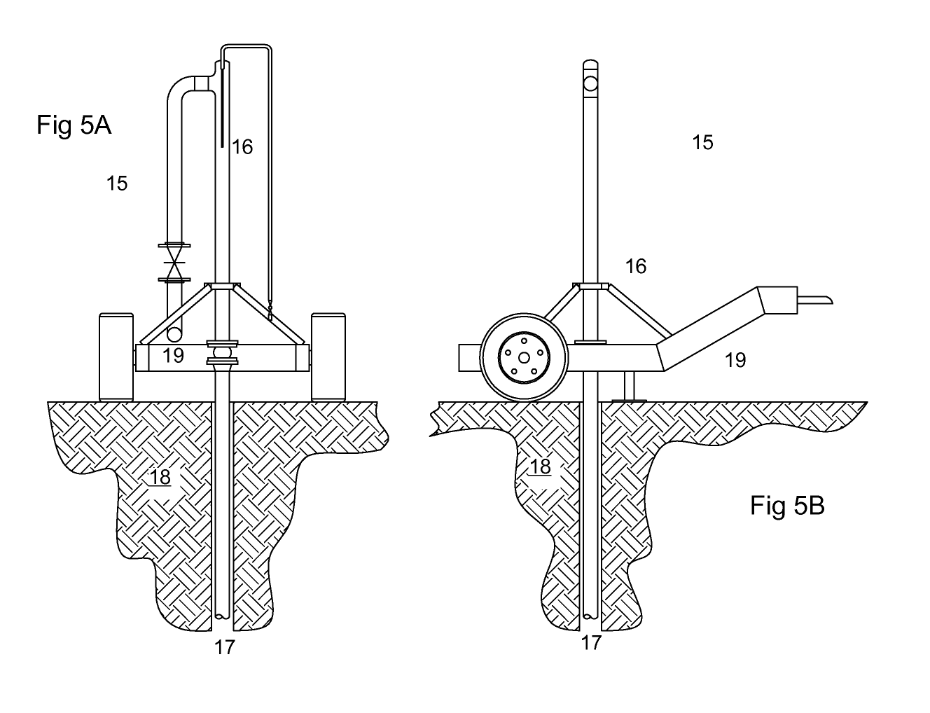

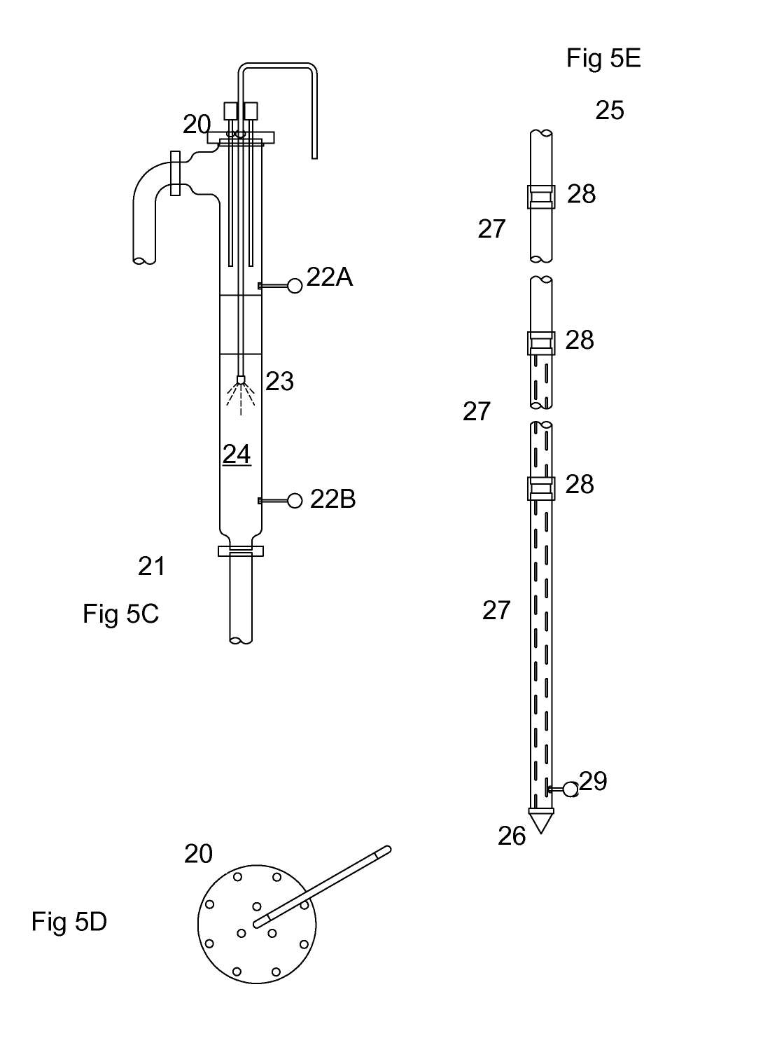

FIG. 5 depicts an exemplary injector, gas and the bacteria/bio-nutrient mixing and injection system 15, including mixer 16 and injector 17 extending into landfill 18 and mounted on a carrier rig 19. However, these components and their respective functions will be described in more detail below with particular reference to the process steps which they perform.

Ingredient Selection of Bacteria, Nutrients, Micronutrients, Alkalinity, and pH Adjustment Compounds, and Special Additives

The first step in evaluation of a potential site is to analyze the available site waste or biomass data. The biomass composition quantity and spatial location will determine the potential energy contained within the area to be treated and the bio vapor injection parameters. If the site has historical gas extraction data, the mass of the amount of gas generated from the site post-placement of waste would be subtracted from the original bio-mass to determine the remaining resource and resulting energy to be potentially extracted. An initial site methane generation model will be developed using a decay curve with Lo, or energy content derived from the waste analysis, and k or rate of conversion, derived from moisture content. The model parameters would be updated with bio-mass core sample analyses over time to determine the degree of digestion and other process parameters associated with the biomass.

Second, the well temperature should be reviewed and a well temperature profile taken to evaluate the operational temperature of the existing biologic process as a function of waste depth. Based upon the temperature recorded, the existing active anaerobic bacteria families can be forecast and appropriate bacteria families selected for injection. Temperature probes will be placed at various intervals in the landfill area to be treated as well as moisture probes to better understand site conditions and confirm anaerobic families and estimate the degree of anaerobic activity. This data is also used to determine the bio vapor injection temperature and spatial openings in the injectors for various bacteria families. Finally, the condensate and/or leachate from the site will be analyzed for its current biological and biochemical properties.

Ingredient Addition to Mixing Tank

Bacteria families can be acquired from natural sources or procured as produced spores on substrates. Two feed sources—one liquid and one solid—could be used to introduce the bacteria into the enclosed mixing tank. Based upon the initial condensate and/or leachate conditions, the nutrient and micro nutrient ingredients will be selected in both composition, quantity, and rate addition.

The mixing tank will be heated up to within a defined temperature of the injector temperature with either a heat exchanger or an electric heater. Solar heat, process heat, direct heat, or electric heaters can be used to maintain the tank temperature. The aqueous solution into which these ingredients are added will be water, or water plus condensate or leachate. A vacuum will be maintained over the mixing tanks to draw off any methane gas formation.

Bacteria Growing Tank

After the bacteria are added to the mixing tanks and filtered, the nutrient and micro nutrient rich buffered solution containing new first-generation bacteria will be transferred to the growing tank. The growing tank contains a large quantity of “neutral” buoyancy, free floating, large surface area, plastic media on which the bacteria family can grow. The dwell time and size of the growing tank is a function of the family of bacteria that is being introduced into the landfill or biomass site. New methane forming bacteria are more efficient at converting solid waste than bacteria that have undergone more than five reproductive generations due to the higher probability of bacteria mutations occurring within the environment. The later bacteria, being influenced by their environment, are referred to as “wild” bacteria. Facultative bacteria reproduce between 15 to 30 minutes, whereas non-facultative methane bacteria can take from one to ten days to reproduce. The growing tank volume (or multiple tanks) would be sized approximately to allow the bacteria to be transferred into the landfill, or biomass waste prior to five generations of bacteria such that the most efficient bacteria for solid waste conversion are being introduced into the vapor stream.

The plastic media on which the bacteria grow are circulated with the tank and as they bump and rub against each other, bacteria colonies are knocked off the plastic films and enter the solution to be pumped to the injector.

Filtration of Bacteria Growing Tank

The heated bacteria growing tank solution will be filtered and transferred to the bio vapor injection system. The filtered product will be fed back into the growing tank for use as growth food for the new bacteria being added from the mixing tank

Nutrient/Bacteria Pumps

The nutrient/bacteria pumps are designed to pressurize the nutrient/bacteria solution in order to move the solution to the injector array. The pump pressure is determined by any hydraulic head and pipe losses that must be overcome in moving the liquid solution to the injectors. This pressure, due to a hydraulic head, is relatively small, and the line losses relatively low due to the relatively low hydraulic head and small volume of solution to be pumped.

Carrier Gas Acquisition, Pressurization, and Heating

The carrier gas for the humidification process can either be landfill gas (CH₄ 56%, CO₂ 44%) or the carbon dioxide resulting from separating the methane in a process facility employed to produce high Btu gas. A blower or compressor would be utilized to collect the carrier gas and pressurize the gas prior to being heated via a heat exchanger and its injection into the landfill. The carrier gas can be heated at a central area via: (1) a process heater, (2) solar energy, (3) a mechanical compression, and (4) electric heater or other means. Alternatively, the carrier gas can be heated at the injector well head via a process heater or other means. Site infrastructure and topography will dictate whether centralized heating or distributed heating would be preferred. Site conditions will dictate the pressure and injection temperature of the carrier gas.

Carrier Gas

The carrier gas will be elevated in temperature above the landfill temperature and humidified with the bacteria enhance, nutrient enriched, buffered solution. The carrier gas for the humidification process can be either biogas with 50% to 56% methane and 40% to 45% CO₂ or 80% to 95% CO₂ with 5% to 10% CH₄ such as the off gas from a high Btu gas processing facility, if one is utilized is on the biomass site.

The methane portion of the carrier gas will pass through solid waste or biomass to a collection well unaltered due to it already being the most reduced form of anaerobic digestion. The CO₂ will serve two functions: (1) It will reduce the hydrogen partial pressure produce by acetate forming bacteria, thus increasing methane production, and (2) it will provide a feed stock for chemolithotrophs to produce methane from carbon dioxide and hydrogen. Therefore, the carrier gas can not only be a source of moving the bacteria/nutrients, etc., but can increase methane production directly as a feedstock.

The introduction of a humidified carrier gas will move the bacteria and nutrients solution more uniformly and further from the injector into the solid waste or biomass matrix prior to being condensed on the waste and, therefore, the carrier gas will facilitate a much more uniform mixing of the bacteria/nutrient/buffer solution with the waste stream, as well as reduced the partial pressure of hydrogen as well as serve as a methane feed stock. This combination of physical attributes of using a humidified carrier gas will provide for a better process control with a minimum of vapor addition to accomplish the waste stream conversion process into methane. Finally, more efficient methane-forming bacteria are being fed continually into the waste matrix with appropriate nutrients, micro nutrients, and buffers providing for a more efficient conversion process than would occur with using existing mutated or wild bacteria for the task.

Carrier Gas Process Variables

A heated carrier gas is preferably used as a vehicle for humidification of a landfill or biomass site. A few examples are provided to illustrate the flexibility of using a carrier gas for water humidification of biomass site.

In order to boost landfill gas production, biologically active vapor can be added to the landfill matrix, increasing the efficiency of the waste conversion to methane. Partial or full vaporization of the bio-water solution is desired to effectively distribute the vapor to the landfill matrix.

The boiling temperature of a bio-water mixture is dependent on the partial pressure of the water vapor, thus in the presence of another gas, the total pressure of the mixture is higher than would otherwise be associated with the corresponding vaporization temperature. For this reason, water exists as liquid and vapor at atmospheric pressure and ambient temperatures, even though the water temperature is well below its boiling point at 14.7 psia (212° F.).

The rate at which evaporation occurs is directly proportional to the heat transfer rate into the fluid. If the body of water is a swimming pool and the air above the pool is both cool and calm, the heat transfer rate will be very low and thus the evaporation rate will be low. In contrast, if the water is being sprayed into a hot vapor line as fine droplets, the evaporation will occur very quickly.

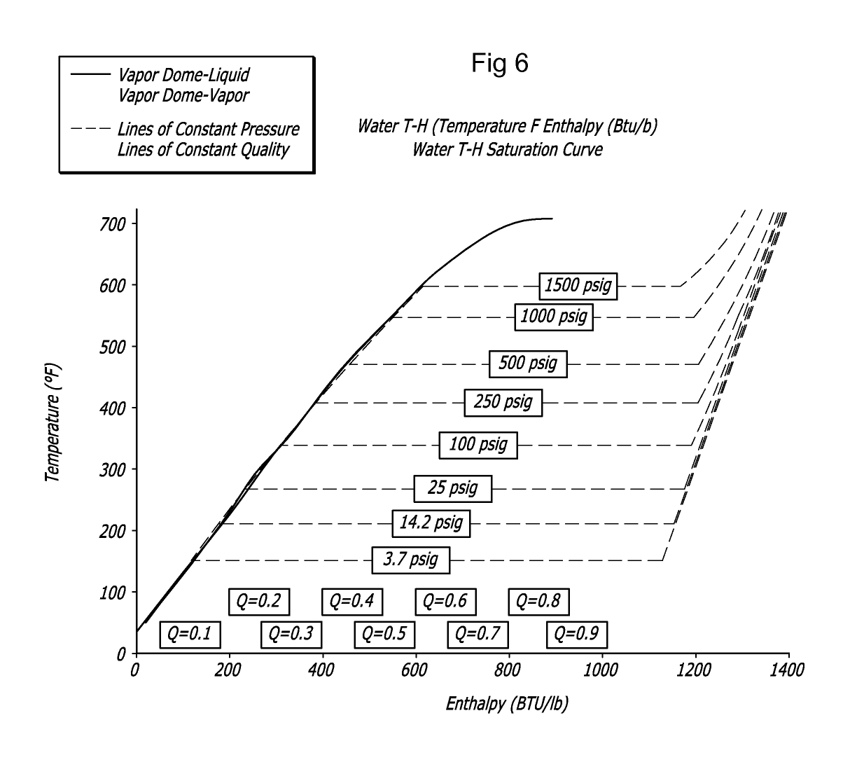

A temperature-enthalpy T-H chart for water is shown in FIG. 6. The large heat of vaporization can be seen in comparison to the sensible heat. For reference, an expansion from the critical point (705.1° F., 3185 psig) to atmospheric pressure would result in a vapor fraction (quality) of 0.738.

Application

The pressure at the injection well in a particular landfill is 15 inches of water vacuum. Injection of only water vapor at 210° F. (the boiling point at 15 inches of water vacuum); injection of a mixture of a gas and bio-water vapor, will be at a lower family of temperatures based on the ratio of gas to water vapor.

Bio-Water and Vapor Injection

Increasing the pressure with a gas or vapor allows the water vaporization to take place at a lower temperature. This is a key to the bio-vapor injection because the temperatures are then low enough to keep the bacteria families in the carrier gas and the biomass matrix alive. Also, the carrier gas can be heated instead of the water providing for a simple vaporization system. The bio-water is then pressure fed through atomizing spray nozzles into the hot carrier gas vapor stream. The small droplets of vapor have a very large surface area to transfer heat and evaporate some or all of the liquid water.

An analysis was performed using both landfill gas (assumed to be 56% Methane and 44% CO₂ by mole) and CO₂ as the carrier gas. The selected injection rate was 2500 gallons per day (1.74 gpm) equivalent of bio-water. The injection pressure was 15 inches of water vacuum (14.16 psia), but the partial pressure of water (3.72 psia) was chosen to correspond to an injection temperature of 150° F. (typical of thermophilic bacteria). The inlet water was assumed to be at 68° F. The “Vapor Fraction” listed in Table 10 is the mass of water vapor divided by the total mass of water.

TABLE 10
Carrier Gas (56% CH₄, 49% CO₂ Temperature, Flow
and resulting Vapor Fraction for an Injection
Temperature of 150° F. and 2500 Gallons/Day

| | Injection | | | | | |
|---|---|---|---|---|---|---|
| | Vapor | Carrier Gas Flow | | | Gas T | Heat Input |
| | Fraction | Ratio | lb/hr | SCFM | ° F. | MMBTU/hr |
| Methane | 0.3 | 1.40 | 1215 | 269 | 847 | 0.347 |
| 56%, | 0.5 | 2.33 | 2025 | 448 | 796 | 0.531 |
| CO₂ 44% | 0.7 | 3.26 | 2835 | 627 | 774 | 0.715 |
| | 0.9 | 4.20 | 3645 | 806 | 762 | 0.899 |
| | 1 | 4.66 | 4050 | 895 | 757 | 0.991 |
| CO₂ | 0.3 | 2.16 | 1878 | 268 | 871 | 0.348 |
| | 0.5 | 3.60 | 3130 | 446 | 815 | 0.532 |
| | 0.7 | 5.04 | 4383 | 625 | 791 | 0.717 |
| | 0.9 | 6.49 | 5635 | 804 | 778 | 0.901 |
| | 1 | 7.21 | 6261 | 893 | 773 | 0.993 |

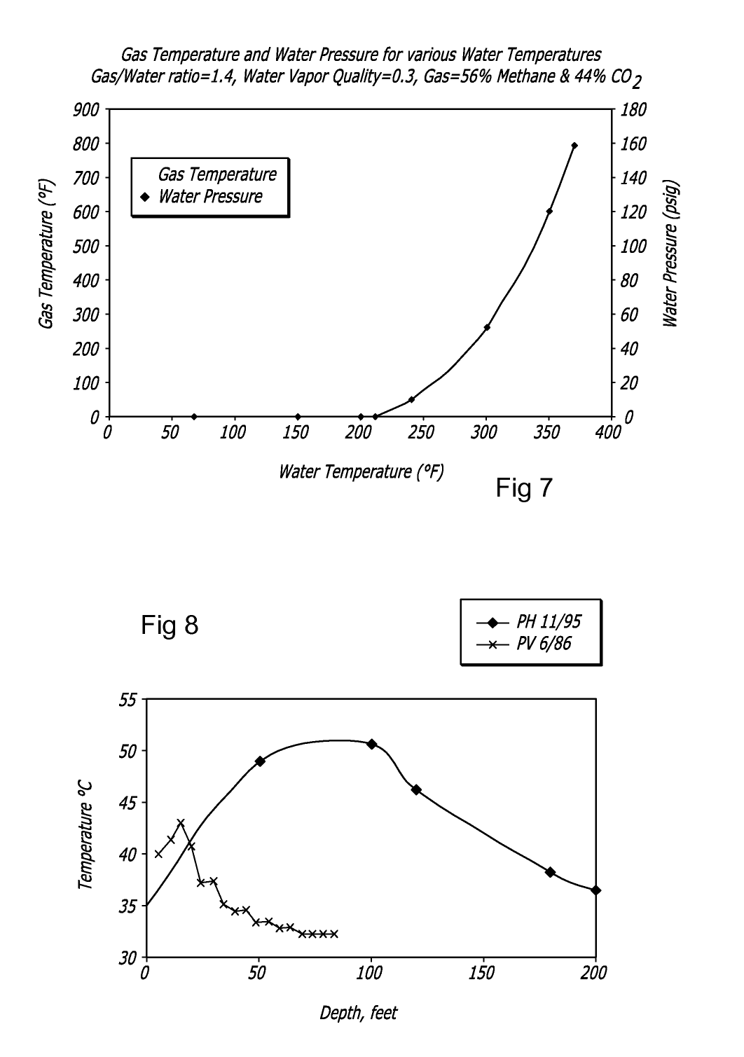

In addition to heating the gas carrier, the water can be pre-heated by solar or geothermal or other means. FIG. 7 shows the reduction in gas temperature as a function of water temperature prior to mixing. The case studied used a gas to water mass ratio of 1.4, which gives a water vapor quality of 0.3 at 150° F. The gas carrier was 56% Methane, 44% CO₂, by mole.

Chemistry of Conversion of Solid Waste

Cellulose comprises a good portion of solid waste. Its decomposition by anaerobic bacteria into methane and carbon dioxide requires a number of intermediate biologic steps, however, the formula on a mass balance basis can be shown as follows:

C₆H₁₀O₅+H₂O=3 CH₄+3 CO₂

In this mass balance, it can be seen that the hydrogen atoms from water end up with the methane gas, and the oxygen from water ends up in the carbon dioxide. Given that this mass balance holds true through the steps associated with the biologic process, water is sharing its hydrogen atoms with methane via biologic anaerobic digestion of cellulose. The water molecules add to the energy value of landfill gas being produced.

In a November 2001 paper comparing electricity producing from RDF and LFG, a theoretical landfill gas production for Ramsey/Washington Counties Resource Recovery Project located in Minnesota was determined using the Counties' waste stream composition. This theoretical conversion formula is presented below.

C₄₁H₆₄O₂₉N+9.75 H₂O→20.875 CH₄+20.125 CO₂+NH₃

Using these two equations as surrogates for solid waste energy potential, we can calculate how much methane and carbon dioxide is produced from 100% biologic conversion of the injected water via biologic processes. This is shown in Table 11.

TABLE 11
Theoretical Methane Produced Per Gallon of Bio-water
Injected at 100% Conversion Efficiency

| | | Methane | CO2 |
|---|---|---|---|
| Pounds of gas | Cellulose | 2.667 | 7.333 |
| per pound of | Theoretical | 1.903 | 5.046 |
| water | | | |
| SCF of gas | Cellulose | 524.0 | 523.4 |
| per gallon of | Theoretical | 374.0 | 360.1 |
| water | | | |

The amount of methane and carbon dioxide required for the Table 11 case of 1.4 gas ratio injection is 0.436 lb and 0.962 lb, per pound of water respectively. The goal of modifying the bacteria, nutrients, micronutrients, alkalinity, etc., of the bio-water converted to vapor for injection, is to approach or achieve 100% conversion efficiency of the injected bio-vapor solution into biomass.

Condensation of Carrier Gas Vapor in Landfill or Biomass

The goal of the bio-vapor stimulation system is to convert more (ultimately all) biomass into biogas and to do so at a faster rate. To accomplish this goal, synergistic families of bacteria need to be deposited on the substrate and then through symbiotic bacterial transformation of the waste and the CO₂ carrier gas to generate more methane in the process of the waste decomposition. To implement this goal, the bacteria families/nutrients/micronutrients/alkalinity adjusted bio-water solution that is used to humidify the site must ultimately condense in microfilms on the waste being treated. The waste temperature profile will provide an indication of the required condensing temperature and an indication of the carrier gas flows and carrier temperature such that the humidified gas will transition into a condensed thin film within the biomass that will be subsequently converted to methane.

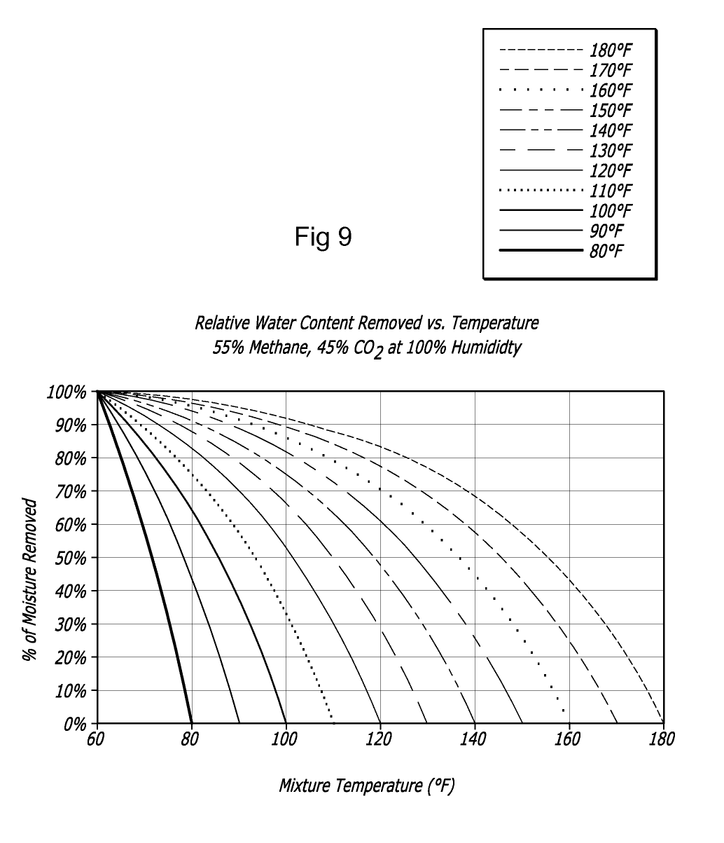

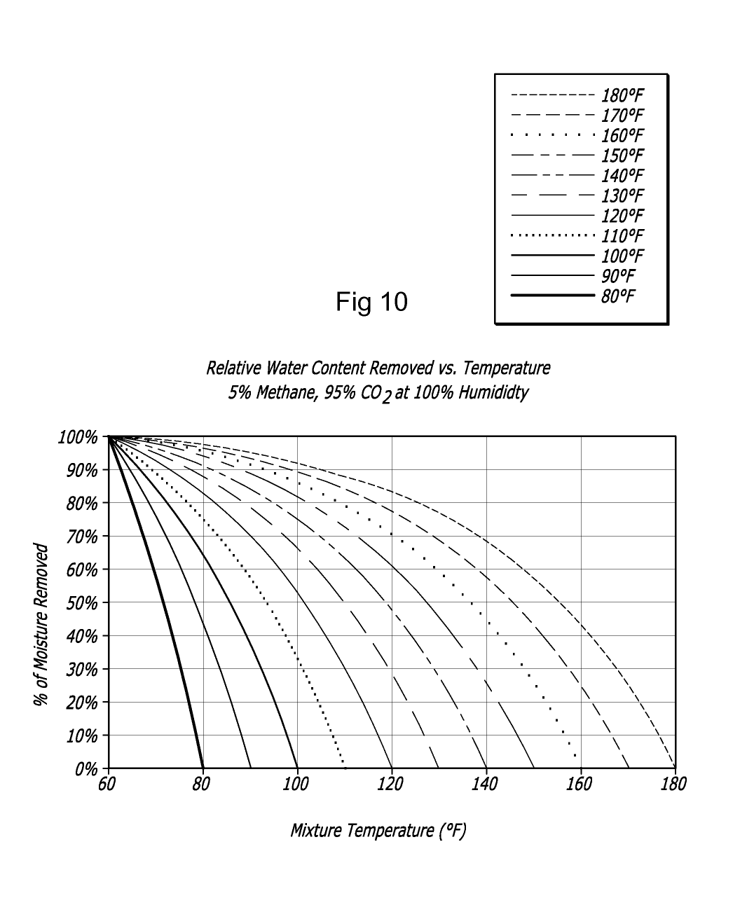

The interior temperature of a landfill reduces in temperature near the landfill surface. FIG. 8 illustrates the temperature profiles of two landfills in California operated by the Los Angles County Sanitation District. The near surface temperatures of these sites as shown are 30° C. or 96° F. and 40° C. or 104° F. FIGS. 9 and 10 illustrate the condensation percentage from two primary carrier gases that have been 100% humidified at the family of temperatures shown. The two carrier gases have almost identical condensation profiles and it can be seen that they will condense from 80% to 98% of their moisture at 95° F., depending upon the initial humidification temperature. Since the gas stream is higher in temperature than the biomass, it will tend to rise within the biomass and, therefore, cool as the waste temperature decreases closer to the surface and condense out its initial humidified water-based, nutrient solution.

Carrier Gas Temperature

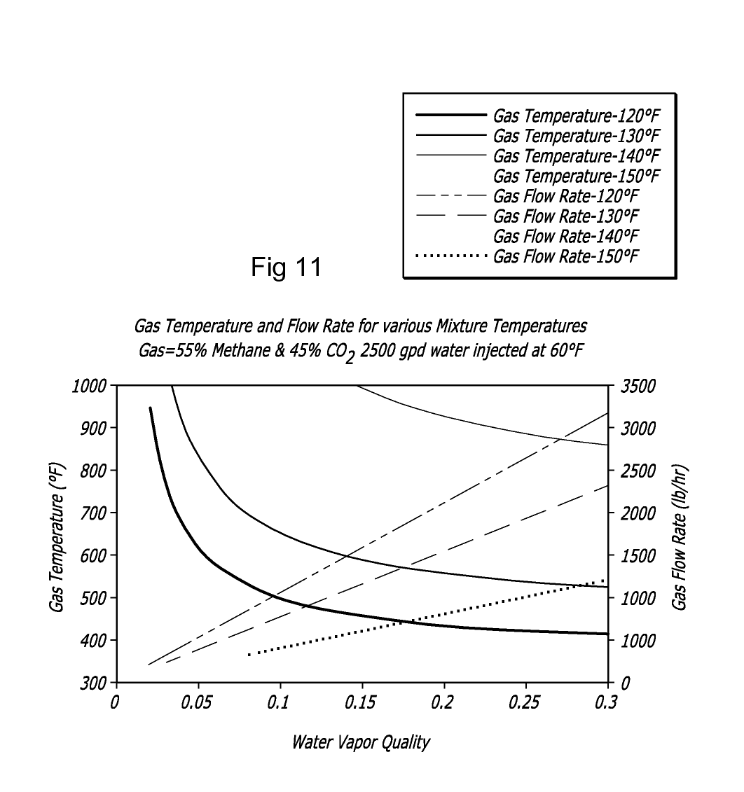

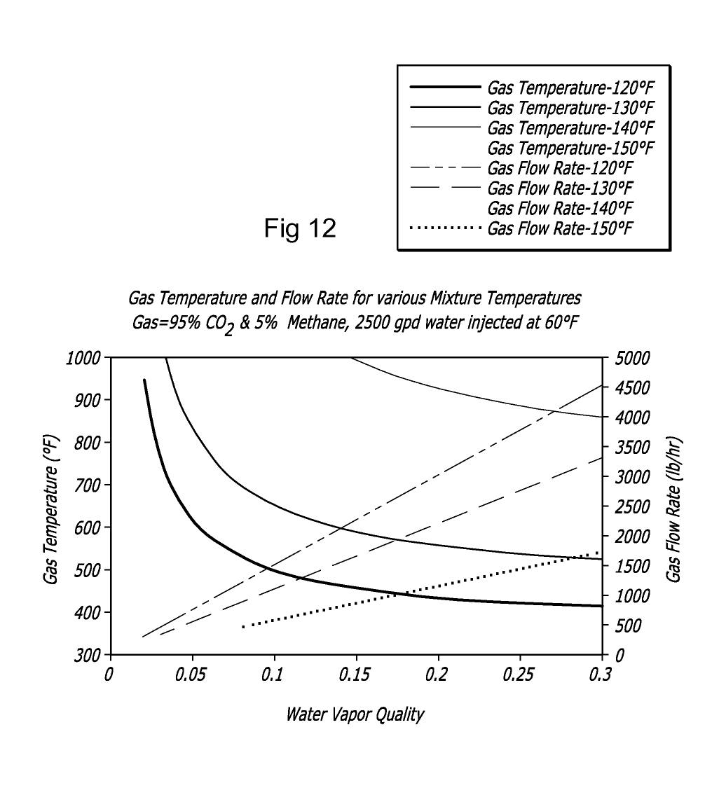

The initial carrier gas temperatures to achieve humidification at reasonable carrier gas flow rates (approximately equal to good gas extraction wells, i.e., 60 to 90 scfm) are shown in FIGS. 11 and 12 for landfill gas and separated or processed (CO₂ enriched) gas, respectively. It can be seen that both carrier gases represent near identical profiles. If the carrier gas flows remain constant, then a lower carrier gas temperature implies a greater misting percentage in the carrier gas and, therefore, a lower vapor percent in the carrier gas being injected into the biomass. The carrier gas temperature prior to the bio-water being introduced and the resulting humidified carrier gas temperature being injected into the biomass site are two key process parameters for the bio-vapor stimulation system technology application.

The landfill temperature profile and the carrier gas humidified temperature going into the biomass, are the parameters from which the families of bacteria going into the mixing tank will be selected, i.e., mesopohiles, thermophiles, or hyperthermophiles.

The other ingredients going into the bio-waste solution will be determined from: (1) collected gas characteristics and condensate analysis from wells collecting the bio vapor stimulated gas production, (2) the initial landfill waste analysis and modeling, and (3) the process sensors in the landfill, and the well data associated with the landfill gas monitoring. These process parameters will be updated from waste core samples used to determine: energy content, percent decomposition, percent moisture, ph, and other waste conditions.

Injection and Mixing Bio-Nutrients with Carrier Gas

Referring now to FIG. 5, the bio-nutrient solution may be introduced into the landfill 18 using a carrier rig mounted injector 17. The injector 17 is used to vaporize the bio-nutrient solution into the hot carrier gas. The hot carrier gas can be either generated at a central location or generated on top 20 of each injector. The injector head 21 has sensors 22A, 22B for the carrier gas temperature going into the injector and the resulting humidified flow going into the landfill, respectively. The humidified flow temperature setting is determined by an analysis of the landfill temperature profile and takes into account the current biologic activity, and the bio-vapor condensation requirements within the biomass.

The bio-nutrients are pumped under pressure and introduced into the injector and the hot carrier gas via a misting nozzle 23. The misting nozzle provides a large surface area for the bio-nutrients solution to be rapidly humidified by the hot carrier gas and injected into the biomass.

Following the humidification section 24 of the injector head 21 the injector is comprised of long multi-section 25 of iron pipe that has been hydraulically penetrated or otherwise inserted with the help of an angled cone 26 at its bottom, into the biomass approximately 75% of the biomass height, with linear slots cut into the pipe along the bottom two thirds of its length. The pipe can be hydraulically inserted into the biomass or placed into a drilled hold into which gravel is packed around the pipe. If the injector is to be hydraulically placed into the site, a hole is first made with a reamer section of pipe slightly larger in diameter than the injector pipe. The sections 27 of the injector pipe are joined with slightly larger section 28 of threaded pipe into which the injector pipe sections are threaded. The hydraulic rig 19 that pushes the reamer and the injector into the landfill matrix should have the ability to be leveled, and to have the pipe angle go into the landfill in a straight fashion measuring the x, y, and z axes of the injector pipe as it is pushed into the biomass.

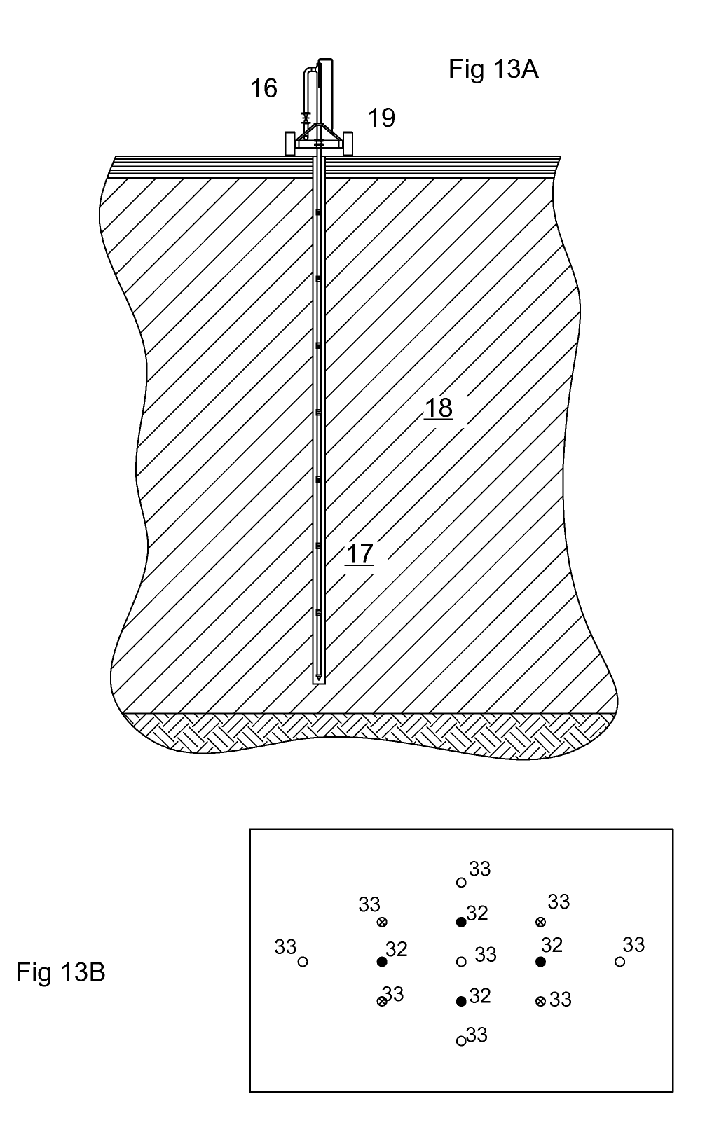

Referring now to FIG. 13, each injector pipe well 32 should be spaced approximately 200 feet from the four closest similarly constructed collector wells 34. The biomass site's porosity could change the injector and collector well spacing. The site's porosity can change with varying forms of waste, cover material, fill practices, compaction methods, etc. However, as shown in FIG. 13B, a grid pattern of four injector wells 32 and nine collector wells 33 would define a modular system for treating and collecting from approximately six acres and would humidify approximately 2,500 gallons of bio-nutrients solution per day. Each well would have a vacuum control valve and a condensate trap such that the bio-vapor stimulated area's COD/BOH, pH, and oxidation-reduction potential (ORP) can be determined from the condensate analysis.

Monitoring Biomass Site for Temperature and Moisture

There are several important measurements that are associated with the biomass site to help evaluate the bio-vapor stimulation flow pattern and process conversion efficiency. The two most important control parameters are the injection temperature of the bio-nutrients solution, the site's temperature profile, and its moisture content. The site's temperature profile is anticipated to be measured by temperature probes located in an array that is similar in design to the injector or collector wells, as well as by taking periodic well temperature measurements at varying depths.

At least three temperatures per injector length should be provided, i.e., bottom, middle, and at the initiation of the slotted portion of the well nearest to the biomass surface. These measurements can be either entered into the control system by an operator or transmitted wirelessly to the process controller (where the data can be subsequently provided on a web-based system). The site's temperature profiles provide inputs into both the bacterial family selection as well as the site's condensation conditions for the bio-nutrients. The temperature profiles could indicate that some injectors will be slotted in a fashion that corresponds to the higher temperature biomass areas within the site which can be fed thermopohilic bacteria/nutrients at their appropriate temperature, and that lower temperatures' biomass areas within the site can be fed mesophilic bacteria/nutrients at their appropriate temperature and condensation parameters. This may also mean that separate mixing and feed tanks may be required with their bio-nutrients based solutions going to their respective injectors and data from the collection injectors providing feedback on their respective segments within the site.

A potentially easier solution may be to provide a different bacteria mix with both mesophiles and thermophilic spores fed into the injectors and the dominate temperature bacteria would grow in their preferred thermal environments.

The moisture sensors 29 would be placed below the injectors' lowest slot to note any changes in the landfill matrix that would require injector slotting modifications, rate of flow changes, etc. These sensors could also be read by an operator and reported or transmitted wirelessly to the central control area.

The injector/well pattern and control system per six acres of biomass is shown in FIG. 13. This initial pattern is designed for a bio-nutrient solution of 2,500 gallons per day being vaporized into the biomass site. As the treated area expands, the injector/well sensor areas will expand.

The process thermal data will be put into a 3-D site computer model which will also use all of the biological data acquired from the condensate and biomass samples. The initial site model will use the biomass composition for energy content and moisture as initial parameters for Lo (energy content) less any losses due to the prior methane generation, and k (rate of gas generation). The k parameters will be initially adjusted such that the site's well flows equals the EPA model using a different Lo. This model using the initial biomass in place and rate of transformation into methane, along with other well based BOD/COD, etc. parameters and site samples, may be used to change/modify process parameters (biology, temperature, etc.) as well as be a tool to evaluate injector pattern and the total biomass conversion as well as biomass conversion efficiency.

Testing Extraction Well Condensate in Injector-Influenced Area for COD/BOD Alkalinity, Nutrients, Etc.

The bio-vapor stimulation process goal is to provide an optimal environment for the anaerobic bacterial conversion of biomass into methane. Because the conversion process to methane is a biologic process, biologic process control parameters are an integral part of the control system.

Table 12 provides the well condensate analytic tests that are to be performed. The frequency of the tests will be determined depending upon the site condition.

TABLE 12
Recommended Analytical Tests for Well Condensate

| | √ Desired Monitoring Frequency | | |
|---|---|---|---|
| Test | Daily | Weekly | As Needed |
| Alkalinity | | | |
| Ammonia-nitrogen | | | |
| Chemical oxygen demand (COD) | | | |
| Gas composition | | | |
| Gas production | | | |
| Grease | | | |
| Organic-nitrogen | | | |
| Orthophosphate-phosphorus | | | |
| pH | | | |
| Settleable solids, supernatant | | | |
| Temperature | | | |
| Total solids | | | |
| Toxicity | | | |
| Volatile acids-to-alkalinity | | | |
| Volatile solids | | | |
| Volume level | | | |

Adjust Process Ingredients and Process Variables if/as Required

The bio-vapor stimulation system has a large number of variables that can be modified to achieve the anaerobic conversion of biomass into methane. At the core of this technology are anaerobic bacteria families that can work together to degrade substrates that other members of the bacteria family utilize to produce methane. Therefore, compatible new bacterial families, their nutrients, micronutrients, with proper alkalinity and temperature provided in a solution which vaporizes and condenses, such that the bacteria families can readily utilize these ingredients to digest the biomass, is at the core of the technology. The distribution of these ingredients within the site in such a fashion that their rate of introduction into the biomass equals the rate of consumption and conversion of the biomass into methane is another key factor of the technology. The bio-vapor stimulation system is designed to address both biological formulations and dispersion patterns of the bio-nutrient solution. The 3-D process control model is designed to provide this data and recommend process modification to the site operator to recommend process modifications that would result in a more optimal conversion environment. This bio-vapor stimulation system is designed to provide landfill stabilization with more methane gas generated from the site, the sequestration of carbon dioxide, an increase in air space, and provide an organic method with the addition of specialized bacteria families to improve gas quality as well as convert more difficult wastes into methane.

1. A bio vapor stimulation process for introducing a bio vapor into a biomass at a waste site, comprising the steps:
selecting at least two types of bacteria and corresponding nutrients for each;
adding the selected bacteria and nutrients into an aqueous mixing chamber to form a bio-nutrient mixture of bacteria and nutrient;
transferring the bio-nutrient mixture of bacteria and nutrient into a temperature controlled tank;
growing the selected bacteria in the temperature controlled holding tank;
filtrating of bacteria from growth chamber effluent;
transporting a bio-nutrient mixture of filtered bacteria and nutrient from the holding tank to an injector via a transfer pump;
injecting the bio-nutrient mixture into the biomass in the vicinity of the injector;
monitoring the landfill site biomass in the vicinity of the injector for temperature and moisture;
testing extraction well condensate in the vicinity of injector for chemical and biologic parameters; and
adjusting process ingredients and process variables to maintain the temperature, moisture and other parameters within a desired range.

2. The bio vapor stimulation process of claim 1 further comprising the step of adding one or more of the following to the bio-nutrient mixture: alkalinity, pure bacteria families, ph additives, and CO2 sequestrian agents.

3. The bio vapor stimulation process of claim 1 further comprising the step of using a heated carrier gas for humidification with subsequent condensation of the bio-nutrient bacteria within the biomass upon cooling of the carrier gas.

4. The bio vapor stimulation process of claim 1 further comprising the step of using thermal sensors within the biomass to determine existing predominant anaerobic bacteria communities for subsequent bacteria families selection to be introduced into bio-vapor stimulation process, as well as to determine vapor condensation temperatures, resulting in the carrier gas temperature for biomass humidification.

5. The bio vapor stimulation process of claim 1, wherein Biologic measurements (COD, BOD) and chemical measurements (ph, N, P) are used as process control variables to adjust nutrients feed, micro-nutrient additive, alkalinity adjustments, temperature control, and bacteria formulations.

6. The bio vapor stimulation process of claim 1 wherein pure bacteria family strains (spores) are activated in the mixing and growing tanks and subsequently humidified into the biomass thereby being more efficient at anaerobically converting the biomass into methane than the site's mutated or wild bacteria.

7. The bio vapor stimulation process of claim 1 wherein the carrier gas, carbon dioxide (plus varying percentages of methane), is sequestered via conversion to methane.

8. The bio vapor stimulation process of claim 1 wherein the carrier gas, carbon dioxide (plus varying percentages of methane) also facilitates the conversion of biomass via anaerobic bacteria by reducing the partial pressure of hydrogen within the biomass allowing the bacteria to operate more efficiently at digesting the carbon in the site.

9. The bio vapor stimulation process of claim 1 wherein special families of bacteria are added to the bio-nutrient solution to perform specific biomass conversion functions such as the elimination of hydrogen sulfide or the digestion of lignin within the biomass.

10. The bio vapor stimulation process of claim 1 wherein the bio-nutrient solution is heated in one or more stages to within 5° F. to 20° F. of the injection temperature to maximize the survival of the bacteria families entering the biomass via vapor injection.

11. The bio vapor stimulation process of claim 1 wherein the injection temperature into the biomass is modified by the ratio of carrier gas to bio-nutrient solution due to the change in partial pressure of the mixture caused by the change n the ratio of carrier gas to bio-nutrient solution.

12. The bio vapor stimulation process of claim 1 wherein the carrier gas is heated in a central area via a process heater, solar energy, waste heat, electric heater, or other means or at the injector via a process heater (gas fired, electric heater, solar heater) on top of the injector.

13. The bio vapor stimulation process of claim 1 wherein the injection temperature of the bio-nutrient mixture is controlled by measuring the carrier gas temperature and the humidified gas temperature after the carrier gas has been mixed with the bio-nutrient solution.

14. The bio vapor stimulation process of claim 1 wherein injection temperature, carrier gas temperature, thermal sensor, moisture sensors, and other process-control signals are transmitted via radio waves (cell phones) or other methods to the process controller for process control modifications.

15. The bio vapor stimulation process of claim 1 wherein process monitoring equipment of thermal sensors, moisture sensors, temperature, carrier gas bio-nutrient ratio, etc., provide inputs into a 3-D model of the biomass area being converted to biogas to ensure process humidification containment and monitor the process conversion's efficiency such that no hydraulic column is formed.

16. The bio vapor stimulation process of claim 1 wherein process signal and the biologic measurement of COD, BOD, ph, etc., is used as inputs to modify (1) bacteria families, (2) nutrients, (3) micro-nutrients, (4) carrier gas temperature, (5) ratio of carrier gas to bio-nutrient solution, and (6) bacteria mixing tank and bacteria growing temperature, etc.,

17. The bio vapor stimulation process of claim 1 wherein bio-nutrient solution is introduced into the carrier gas in the injector via one or more atomizing nozzles to ensure rapid vaporization.

18. The bio vapor stimulation process of claim 1 wherein a family of injector and collector wells is placed into the biomass matrix at approximately 100 foot centers. Via a piping modification on the biomass site, the injector wells can become collector wells and the collector wells can become injector wells thus providing a more thorough mixing of the bio-nutrient solution in the carrier gas with the biomass.

19. The bio vapor stimulation process of claim 1 wherein the injectors inject the mixture of carrier gas and bio-nutrient mixture by means of injector wells constructed of steel with a vertical pattern of 3″ long×⅛″ width slots extending through the bottom two-thirds of the well length and with small sections of pipe with circular holes to facilitate the vapor injection pattern into the biomass matrix.

20. The bio vapor stimulation process of claim 1 wherein a hole pattern in the injectors is slotted to be area specific, to accommodate the injection of temperature predominant bacteria species into a specific area.

21. The bio vapor stimulation process of claim 1 wherein a swirl vane is added at the top of the injector to put a spin on the injector vapor, resulting in a different trajectory on the carrier gas as it exits the injector slots which will also aide in the mixing of the carrier gas and bio-nutrient solution into the biomass matrix.

22. The bio vapor stimulation process of claim 1 wherein at least three thermal sensors are be placed inside injector pipes to measure the biomass matrix temperature at various depth.

23. The bio vapor stimulation process of claim 1 wherein a small amount of dirt is put at the bottom of monitoring wells having a depth is at least 10 to 20 feet below the injector wells into which a moisture sensor is placed to ensure that the process is not creating a hydraulic column, and which are slotted only in the bottom one to two feet.

24. The bio vapor stimulation process of claim 1, further comprising the steps:
pressurizing a carrier gas;
adding heat to the carrier gas; and
mixing the heated carrier gas with the bio-nutrient mixture in the injector.

25. The bio vapor stimulation process of claim 1, wherein:
the chemical and biologic parameters include pH, chemical oxygen demand (COD), and biologic oxygen demand (BOD).

26. The bio vapor stimulation process of claim 7, wherein the conversion to methane is performed by anaerobic bacteria.
